(19) United States
(12) Patent Application Publication
(10) Pub. No.: US 2016/0030769 A1
(43) Pub. Date: Feb. 4, 2016

(54) METHOD AND DEVICE FOR FAST RASTER BEAM SCANNING IN INTENSITY-MODULATED ION BEAM THERAPY

(71) Applicants: John M. Cameron, Bloomington, IN (US); Vladimir Anferov, Bloomington, IN (US); Steven Vigdor, Bloomington, IN (US)

(72) Inventors: John M. Cameron, Bloomington, IN (US); Vladimir Anferov, Bloomington, IN (US); Steven Vigdor, Bloomington, IN (US)

(73) Assignee: PHENIX MEDICAL LLC, Bloomington, IN (US)

(21) Appl. No.: 14/450,070

(22) Filed: Aug. 1, 2014

(51) Int. Cl. A61N 5/10

(52) U.S. Cl. CPC A61N 5/1043; A61N 5/1071; A61N 5/1068; A61N 2005/1087

(57) ABSTRACT

A method and device are designed to deliver intensity-modulated ion beam therapy radiation doses closely conforming to tumors of arbitrary shape, via a series of two-dimensional (2-D) continuous raster scans of a pencil beam, wherein each scan takes no more than about 100 milliseconds to complete. The device includes a fast scanning nozzle for the exit of an ion beam delivery gantry. The fast scanning nozzle has a fast combined-function X-Y steering magnet, and is coupled to a rastering control system capable of adjusting the length of each scan line, continuously varying the beam intensity along each scan line, and executing multiple rescans of a tumor depth layer within a single patient breathing cycle. An in-beam absolute dose and dose profile monitoring system is capable of millimeter-scale position resolution and millisecond-scale feedback to the control system to ensure the safety and efficacy of the treatment implementation.

### FIELD OF THE INVENTION

This invention generally relates to a method and system for radiotherapy treatments. More particularly, the present invention relates to particle beam delivery for ion beam therapy, based on the scanning of intense accelerator beams of small cross-sectional area (so-called “pencil” beams), and of adjustable energy and intensity, to irradiate the full volume of an arbitrary-shaped target tumor conformally, while providing minimal dose to surrounding healthy tissue.

### BACKGROUND OF THE INVENTION

In comparison to standard X-ray therapy, proton or heavier-ion beam therapy is capable of significantly improving dose localization by increasing dose delivered to the target volume while minimizing dose delivered to the surrounding tissue. These improvements are based on the finite penetration range of therapeutic ion beams in the target material. Furthermore, the energy deposition to the target material increases as the ion beam slows down and reaches a sharp maximum near the end of the penetration range. As a result, ion beam therapy has the potential to provide the best possible treatment option for control and elimination of tumors, with fewer short- and long-term toxic side effects.

The majority of ion beam therapy treatments to date have been delivered using legacy passive scattering systems, wherein the treatment dose field is formed through patient-specific apertures and range compensators. However, the inherent advantages of ion beam therapy are best exploited by an alternative approach, applying pencil beam scanning (PBS) methods of dose delivery to achieve full 3-D conformity to any tumor volume without using apertures and compensators. Pencil Beam Scanning refers to a method where a small diameter incident ion beam is spread laterally across the tumor at a certain depth using scan magnets that sweep the beam in two lateral dimensions. The scan magnets are situated near the exit of a beam delivery gantry that can be rotated to irradiate the tumor from multiple directions. The beam intensity is varied for each 3-D spot (voxel) to achieve a dose distribution that conforms exactly to the tumor area at that depth. Repeating this process for a range of decreasing energies (energy stacking) allows treatment of the full tumor volume with any arbitrary shape. The beam intensity is varied for each 3-D spot (voxel) to achieve a dose distribution that conforms exactly to the tumor volume. The passive scattering and PBS approaches are contrasted schematically in FIGS. 1 and 2, and in realization of treatment plans for a given tumor in FIG. 3.

The beam intensity modulation that can be realized with the PBS technique allows ion beam therapy to compete favorably with intensity-modulated radiation therapy (IMRT) carried out with X-rays. The advantages of PBS are both clinical and financial. Some of these advantages are discussed hereinbelow.

For example, target volumes of arbitrary shapes can be irradiated with a single dose field (gantry angle setting). This feature of PBS brings multiple benefits. Double scattering and uniform (without intensity modulation) scanning systems conform the distal edge of the dose distribution to the target shape, but inevitably generate areas of excessive dose to healthy tissue proximally, as indicated in FIG. 1 and the left-hand frames of FIG. 3. Thus, PBS improves conformity of an ion beam dose delivered to the target. Furthermore, the improved conformity of a single PBS field allows one to obtain required target coverage with fewer fields, which simplifies the entire treatment and reduces the total treatment cost. In cases of complex shape tumors, PBS is expected to reduce the need for dose field matching and patching.

The secondary neutron dose to the patient is reduced. Due to the avoidance of first and second scatterers, collimators and compensators, the beam has fewer nuclear interactions in material close to the patient, resulting in a great reduction of secondary neutron dose to the patient. While several studies have found the neutron dose in proton therapy to be small, the high relative biological effectiveness of neutrons warrants reduction of the neutron dose to as low a level as possible, especially for pediatric treatments.

The elimination of patient-specific devices results in substantial savings in cost and treatment time. PBS eliminates the need to produce and dispose of activated patient-specific devices and eliminates the time required to install them, verify their match with the treatment field and assure their correct positioning with respect to the target isocenter. It also removes the need to change patient-specific devices between dose fields. Those changes require entry to the treatment room and the patient-specific devices are often too heavy for one therapist or radiological technician to handle.

The promise of PBS has led to predictions of rapid near-term growth in the number of ion beam therapy clinics worldwide and in the fraction of radiation treatments that will be delivered via ion beams. These projections assume that the technology to enable PBS will be available at a reasonable cost, and that techniques will be developed to overcome remaining limitations on its applicability. Indeed, intensity-modulated proton therapy (IMPT) treatments are already available at several operating clinics (examples are the Paul Scherrer Institute in Switzerland and the Cadence Health Clinic in Warrenville, Ill., U.S.A.), where they are being used for an increasing fraction of treatments. The particular implementation of PBS to date has been based on so-called spot beam scanning (SBS).

Details regarding pencil beam scanning and spot beam scanning are disclosed in the following three patent references: U.S. Pat. No. 8,541,762, “Charged Particle Irradiation Device and Method”, issued on Sep. 24, 2013; PCT Publication No. WO2013149945, “A System for the Delivery of Proton Therapy by Pencil Beam Scanning of a Predeterminable Volume Within a Patient”, published on Oct. 10, 2013; and U.S. Pat. No. 8,586,941, “Particle Beam Therapy System and Adjustment Method for Particle Beam Therapy System”, issued on Nov. 19, 2013; the entire teachings and disclosures of which are incorporated herein by reference thereto.

In the conceptually simplest version of the SBS approach, each 3-D voxel in the tumor volume is irradiated until it receives its full intended dose, after which the beam is moved to irradiate the next voxel in the same depth layer. Under normal clinical conditions, “painting” a single depth layer in the target tumor may then take several seconds to complete, before the beam energy is reduced to perform an analogous scan on the next, less deep, layer.

There are several limitations unique to beam scanning techniques, with some of these limitations, discussed below, exacerbated by the above-described SBS approach.

1) The spot-to-spot scanning approach is more sensitive to organ motion than passive scattering. Spot-to-spot scanning is described in “Moving Target Irradiation With Fast Rescanning and Gating in Particle Therapy”, Takuji Furukawa et al., Med. Phys. 37, 4874 (2010); and also described in “A Study on Repainting Strategies for Treating Moderately Moving Targets With Proton Pencil Beam Scanning at the New Gantry 2 at PSI”, S. Zenklusen et al., Phys. Med. Biol. 55, 5103 (2010), the entire teachings and disclosures of which are incorporated herein by reference thereto. The interplay between the scanned beam motion and the target motion may result in localized under-dosage in parts of the target volume and over-dosage in other parts of the target volume or in the surrounding tissues, as indicated by simulations in FIG. 4 and by measurements in FIG. 5. Medical device companies, IBA and Varian, have adopted two techniques to mitigate target motion effects in spot beam scanning. The beam can be gated off when patient movement is sensed or anticipated, or the full dose to a given depth layer can be delivered in two or more “repaints,” rather than in a single 2-D scan. However, the concern remains that such repainting is done on a time period of about 1-2 seconds and could still interfere with target motions due to patient breathing, which has a typical period of 3-4 seconds.

2) High sensitivity to beam misalignment. Even small beam misalignment of a few millimeters can cause significant dose perturbations when non-uniform dose distributions are combined from several fields. Varian scanning systems are mitigating this by improving beam spot positioning, while IBA protocols involve a test shot that delivers a small fraction of the prescribed dose prior to each treatment layer to measure the misalignment and recalculate the dose map accordingly. Both of these methods increase the overall treatment time.

3) Pencil beam scanning is sensitive to the scanning accuracy. A high degree of accuracy and robustness is required from the scanning system since, for example, a failure to move to the next beam spot would result in 100% spot overdose in about 10 milliseconds. Both Varian and IBA experienced scanning distortion at large spot displacement and developed expensive custom-made scanning controllers to monitor scanning accuracy.

4) Large spot-to-spot dose variation requires precision dose rate control and dose measuring electronics with large dynamic range. When dose is delivered to the proximal target layers some spots will have already received a large fraction of their required dose during delivery to distal layers. This can generate large variation in dose per spot required within a given layer. Delivery of low dose spots is a challenging task for present dosimetry electronics and IBA has imposed a low dose limitation on its SBS system, which could preclude delivery of proton boost or patch fields at 40 centigray or below.

Embodiments of the invention provide a method and system for radiotherapy treatments that addresses the issues raised above. These and other advantages of the invention, as well as additional inventive features, will be apparent from the description of the invention provided herein.

### BRIEF SUMMARY OF THE INVENTION

In a particular aspect, embodiments of the invention provide a fast scanning nozzle for an ion beam therapy gantry, comprising a scanning system and a dose monitoring system that enable intensity-modulated dose delivery to a tumor of arbitrary shape in a sequence of multiple repaints, each delivering a fraction of the dose intended for a given depth layer in a time interval much shorter than typical organ motion periods. The invention is based on a combined function X-Y scanning magnet capable of continuously moving the beam spot across a predetermined 2-D raster scan pattern, at speeds exceeding 25 meters per second. The scanning control system is capable of varying the length of each scan line, and of continuously varying the beam intensity along each scan line, to achieve conformal irradiation of complex dose field shapes. The dose monitoring system measures the position, length and intensity distribution of each scan line, and applies those measurements for feedback corrections to beam position and intensity on millisecond time scales. A complete 2-D painting of a depth layer can then be achieved in a time interval of less than 100 milliseconds, during which the target tumor will be essentially stationary. As many as 10-20 repaints of the depth layer can be completed, as needed, within a single patient breathing cycle.

In one aspect, embodiments of the invention provide a method for irradiating a target volume with a charged particle pencil beam. The method includes the steps of continuously scanning the pencil beam of charged particles over a two-dimensional (2-D) raster scan pattern, applying length-variation for each scan line to conform to the 2-D raster scan pattern at a given depth, applying pencil-beam-intensity variation along each scan line, and completing multiple pencil beam scans of the entire 2-D raster scan pattern for each target depth layer of the target volume.

In a particular embodiment, the method includes pausing the scanning upon completion of the scanning of each target depth layer in the target volume, and changing the energy value of the pencil beam prior to scanning a next target depth layer. The method may also include measuring the position, length, and intensity distribution of each scan line, and using the measurements to make feedback corrections to pencil beam position and pencil beam intensity for scanning subsequent scan lines or for subsequent repaints of the entire 2-D raster scan pattern.

In certain embodiments, the method requires scanning the pencil beam along a scan line at a speed of at least 25 meters per second. In a further embodiment, the method calls for scanning the entire 2-D raster scan pattern for a given depth layer in 100 milliseconds or less, with the full dose for that depth layer possibly to be delivered in multiple repaints of the 2-D raster scan pattern. The method may also include gating the pencil beam on and off for the multiple repaints of the 2-D raster scan pattern, wherein the gating is timed with respect to a patient's breathing cycle. Some embodiments of the method include continuously scanning the pencil beam of charged particles over a two-dimensional (2-D) raster scan pattern using a fast scanning nozzle with scanning magnet.

In a particular embodiment, the method includes measuring a dose distribution as a function of position along each scan line such that a measurement of absolute dose is accurate to within 2%, and a measurement of pencil beam spatial position is accurate to within two millimeters. The method may call for synchronizing scanning of the pencil beam with the measuring of dose distribution. In other embodiments, the method includes interrupting pencil beam operation if the absolute dose measurement indicates that an actual dose delivery is outside of a predetermined range of acceptable values.

In certain embodiments, the method requires monitoring electric current drawn by the scanning magnet, monitoring magnetic field strength of the scanning magnet, monitoring patient position with respect to pencil beam position, and discontinuing pencil beam operation if any one of the electric current, magnetic field strength, and patient position deviates from a predetermined range of acceptable values.

In another aspect, embodiments of the invention provide a system for delivering targeted ion beam therapy to a target volume. The system includes a fast-scanning nozzle for targeting an ion beam. The fast-scanning nozzle having a scanning magnet is configured to deflect the ion beam in two dimensions. A scanning magnet controller is configured to control the fast-scanning nozzle to provide continuous scanning of the ion beam over a 2-D raster scan pattern at a first target depth layer of the target volume such that multiple scans of the 2-D raster scan pattern are performed. The scanning magnet controller is further configured to control the fast-scanning nozzle to make multiple ion-beam scans of 2-D raster scan patterns for each of a plurality of target depth layers of the target volume other than the first target depth layer.

In certain embodiments, the fast-scanning nozzle and scanning magnet are configured to deflect the ion beam in two perpendicular lateral dimensions such that the two perpendicular lateral beam deflections have identical source-to-axis distances. Further, the fast-scanning nozzle may include a nozzle housing surrounding the scanning magnet. The nozzle housing has an ion beam entry window at a first end of the housing, and an ion beam exit aperture at a second end of the housing opposite the first end. In particular embodiments, the ion beam exit aperture is disposed in a retractable housing projection. The retractable housing projection may include a holder for patient-specific apertures and compensators.

In a particular embodiment, the fast-scanning nozzle has a beam monitoring ionization chamber adjacent to the ion beam entry window. The beam monitoring ionization chamber is configured to measure the size, position, and intensity of the ion beam after it passes through the ion beam entry window, and to provide the measurement data to the scanning magnet controller. The scanning magnet controller may be configured to make feedback corrections to ion beam position and intensity based on the measurement data from the beam monitoring ionization chamber. In some embodiments, the fast-scanning nozzle includes a dose monitoring chamber downstream of the scanning magnet and upstream of the ion beam exit aperture. The dose monitoring chamber is configured to provide data, regarding dose delivery and ion beam spatial position, to the scanning magnet controller.

In certain embodiments, the dose monitoring chamber includes a position-sensitive array of gaseous ionization chambers, or a gaseous tracking detector coupled to position-insensitive ionization chambers, or a scintillation detector with position-sensitive readout. One or more sensors may be disposed in the nozzle housing proximate the dose monitoring chamber. The one or more sensors are configured to sense one of temperature, humidity, and pressure. In at least one embodiment, the fast-scanning nozzle includes a light projection mirror disposed in the nozzle housing downstream from the dose monitoring chamber. The light projection mirror is configured to align the fast scanning nozzle with the target volume.

The system may further include an energy modulation unit configured to vary the energy of the ion beam before it enters the fast scanning nozzle. In an embodiment of the invention, the scanning magnet controller controls a safety interlock configured to shut off the ion beam if a dose measurement indicates that an actual dose delivery is outside of a predetermined range of acceptable values, and also configured to shut off the ion beam if any of one or more sensors, monitoring one of electric current drawn by the scanning magnet, magnetic field strength of the scanning magnet, and patient position with respect to pencil beam position, senses that one of the electric current, magnetic field strength, and patient position is outside of a predetermined range of acceptable values.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a schematic plan view of a conventional passive scattering system for delivery of an ion beam of fixed energy and intensity to irradiate a tumor with the aid of patient-specific apertures and compensators;

FIG. 2 is a schematic plan view of a pencil beam scanning system for delivery of an ion beam of variable energy and intensity to irradiate a tumor;

FIGS. 3A and 3B are illustrations showing a comparison of treatment plans for two different approaches to proton therapy dose delivery for a tumor of complex shape wrapped around a critical organ;

FIG. 4 is an illustration showing exemplary simulated dose perturbations that may result from the interplay between spot beam scanning and target motion frequencies;

FIG. 5 is an exemplary illustration of a radiographic record of the net dose delivery in a proton spot beam scan for which the film was moved laterally back and forth through a water phantom to simulate organ motion inside a patient;

FIG. 6 shows a schematic layout of one embodiment of the fast scanning nozzle, comprising a combined function X-Y scanning magnet and a dose monitoring system with two-dimensional position measurement capability, configured to be embedded at the end of a rotatable beam delivery gantry; and

FIG. 7 is a schematic diagram illustrating components of a fast scanning nozzle control system, comprising a scanning controls module with dedicated safety controller and a dose monitoring controls module, according to an embodiment of the invention.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Embodiments of the present invention are directed to providing a cost-effective alternative to Spot Beam Scanning that is capable of delivering Intensity-Modulated Ion Beam Therapy via a sequence of fast, moderate-dose repaints, with substantial mitigation of the above-described problems related to organ motion and beam misalignment. Embodiments of the invention also facilitate an approach to ion beam therapy that relaxes some of the demands on monitoring dosimetry implied by concerns related to scanning accuracy and spot-to-spot dose variation.

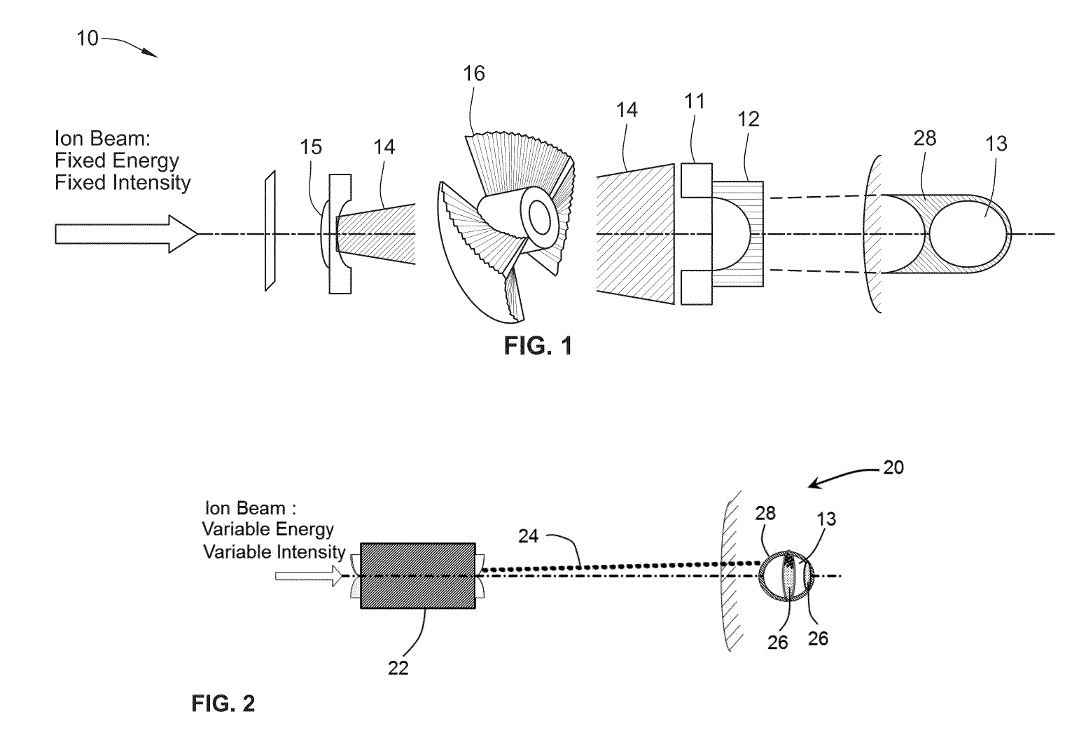

FIG. 1 shows a schematic layout of a conventional fixed-energy, fixed-intensity passive scattering system 10 for ion beam therapy delivery to a target volume, or tumor 13, using patient-specific apertures 11 and compensators 12 to form the desired radiation field from the broad beam 14 produced via scattering foils 15 and a range modulator 16. For comparison, FIG. 2 shows a system 20 for delivery of variable-energy, intensity-modulated ion beam therapy, using a scanning system 22 to scan a pencil beam 24 across depth layers 26 of a tumor. Two representative depth layers 26 are indicated in the figure. The shaded areas 28 in FIGS. 1 and 2, schematically indicating dose distributions outside the tumor volume, illustrate how pencil beam scanning can lead to reduced irradiation of healthy tissue adjacent to the tumor.

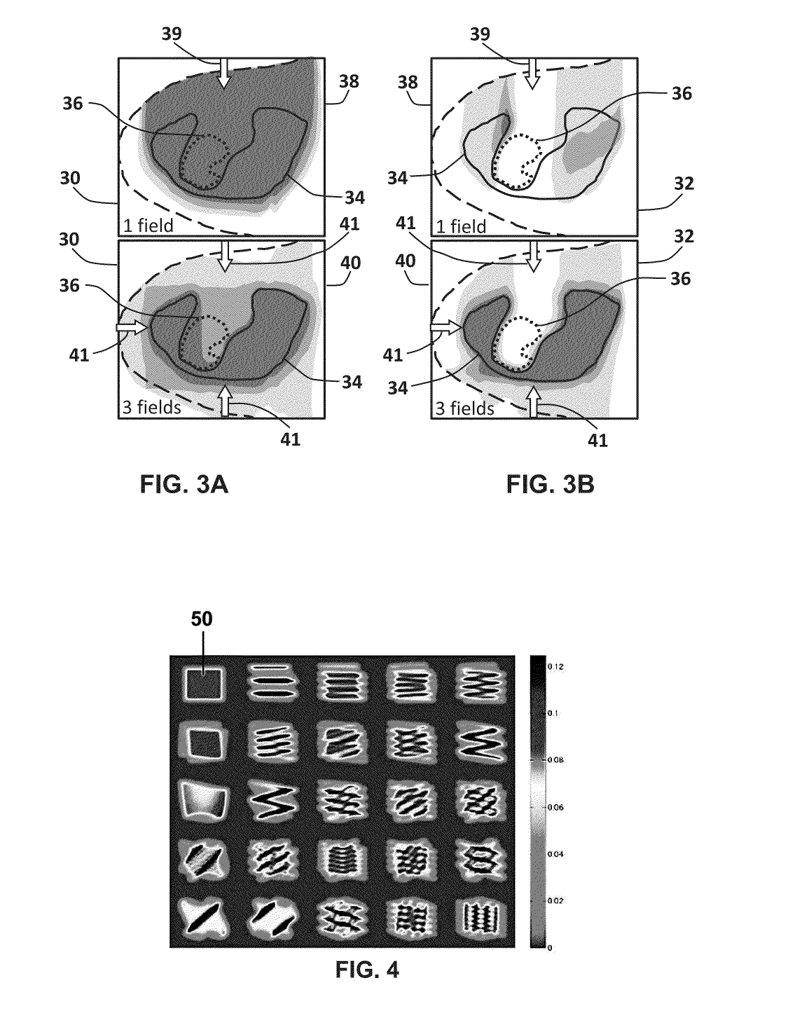

FIGS. 3A and 3B compare proton therapy dose delivery plans for the same two approaches compared schematically in FIGS. 1 and 2. Similar proton therapy dose delivery plans are discussed in “An Overview of Compensated and Intensity-Modulated Proton Therapy”, A. J. Lomax, American Association of Physicists in Medicine (AAPM) Summer School (2003), the entire teachings and disclosure of which is incorporated herein by reference thereto. The left-hand frames 30 of FIG. 3A illustrate the dose strength that would be delivered by a passive scattering treatment, while the right-hand frames 32 of FIG. 3B illustrate that for a pencil beam scanning modality, in both cases for the same tumor 34 of complex shape wrapping around a critical organ 36. In each case, the upper frames 38 show shaded dose intensity contours that can be attained with a single dose field (with beam incident in the direction indicated by the arrow 39), while the lower frames 40 show shaded dose contours attainable with three distinct dose fields, delivering beam in successive treatment stages along the directions indicated by the three arrows 41. The darkest shading of the contours in all four frames corresponds to a high delivered dose, and the lightest shading to a low delivered dose. Regions with no shading receive negligible doses. FIG. 3 clearly illustrates the promise of pencil beam scanning for sparing healthy critical organs adjacent to complex tumors from excessive radiation dose.

FIG. 4 (adapted from T. Furukawa, et al., Med. Phys. 37, 4874 (2010)) shows exemplary simulated dose perturbations resulting from the interplay between spot beam scanning and target motion frequencies. The square 50 in the top left corner shows the uniform dose that would be delivered to a stationary target, while the other images show the dose to target resulting from the same spot beam scan under various conditions of target motion.

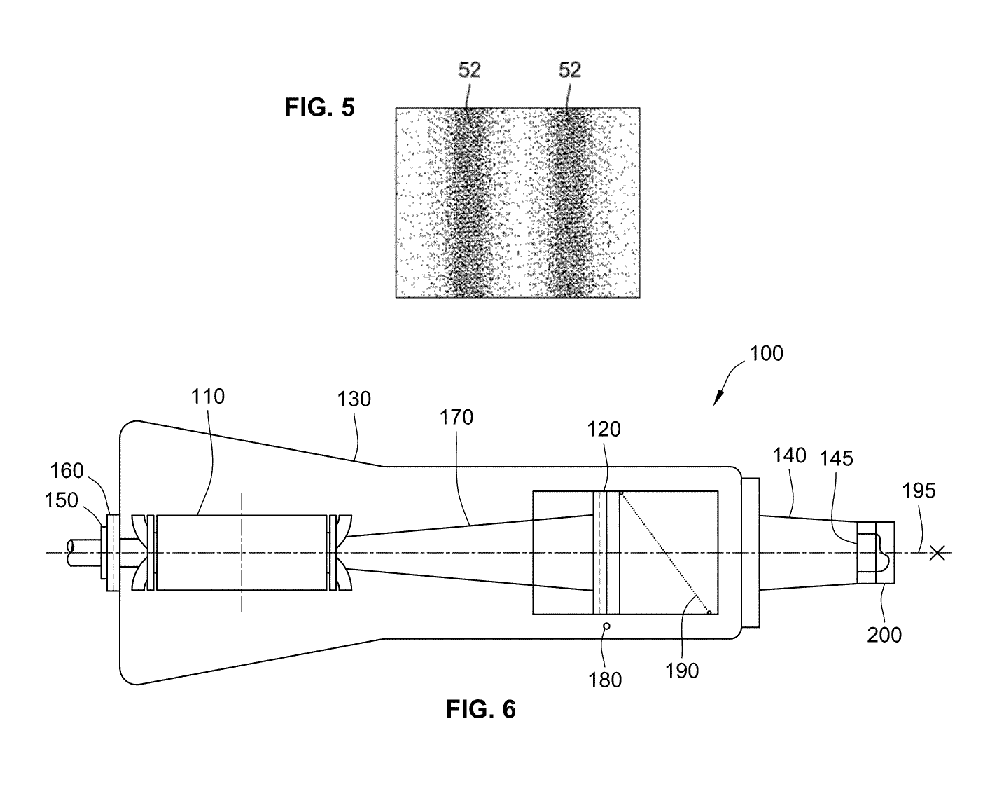

FIG. 5 shows a graphical representation of exemplary experimental results that provide qualitative confirmation of the dangers of organ motion illustrated by the simulations in FIG. 4. In particular, FIG. 5 shows a record on radiographic film of the net dose delivery in a proton spot beam scan for which the film was moved laterally back and forth through a water phantom with a four-second period, comparable to a typical patient breathing period. The dose was delivered over a 10 cm×10 cm area in 43×43 voxels, with dose delivery to each voxel lasting for approximately six milliseconds, a typical duration for clinical spot beam scans. The vertical stripes 52 seen in the figure represent ˜50% variations in dose resulting from the interplay of target and beam motion, illustrating potential complications introduced by organ motion for spot beam scanning treatments. When the film was held stationary, the same beam scan produced a uniform dose within a 10 cm×10 cm area. The proposed solution for such potential problems is to utilize the present invention to perform a complete 2-D beam scan over time intervals much shorter than patient breathing periods.

Referring now to the invention in more detail, in FIG. 6 there is shown a schematic view of one possible embodiment of the invention, in which the fast scanning nozzle 100 comprises an X-Y scanning magnet 110 and a dose monitoring chamber 120 housed in a lightweight nozzle frame 130 with a retractable snout 140 that includes an ion beam exit aperture 145. The ion beam enters the nozzle from the gantry through a vacuum window 150 and a beam monitoring ionization chamber 160, and is transmitted through the scanning magnet 110 to the dose monitor 120 in a section 170 that is held either at vacuum or filled with helium, in order to minimize beam scattering through air. In order to improve accuracy of the dose monitoring chamber readout, a set of sensors 180 is installed in its vicinity for the measurement and recording of ambient air temperature, pressure and humidity.

Optionally, the fast scanning nozzle 100 can also include a light projection mirror 190 useful for initial alignment of the patient to the nozzle axis 195 and a holder 200 for patient-specific apertures and compensators mounted to the retractable snout 140. Even though they are superfluous for the majority of patients treated via pencil beam scanning, the apertures and compensators 200 can provide optimal additional passive protection for critical organs that may lie immediately adjacent to the planned radiation field. The fast-scanning nozzle 100 and scanning magnet 110 may be configured to deflect the ion beam in two perpendicular lateral dimensions such that the two perpendicular lateral beam deflections have identical source-to-axis distances.

In more detail, still referring to FIG. 6, the beam-monitoring ion chamber 160 measures the size, position and intensity of the ion beam entering the nozzle, information that will be used for feedback loops controlling the beam centering and intensity. The X-Y scanning magnet 110 deflects the beam according to the specified scan profile to cover the full target tumor area. The use of combined X-Y magnet coil geometry provides identical source points for the beam deflection in the two lateral dimensions, thereby simplifying treatment planning and improving agreement between planned and generated dose distributions.

The dose monitoring chamber (DMC) 120 provides redundant signals on total dose delivered to the target as well as information on the dose profile and its conformity to the target shape. In order to meet clinical acceptance criteria, the DMC 120 must be capable of measuring absolute dose with 1-2% accuracy as a function of 2-D position measured with 1-2 mm spatial resolution, and to deliver output signals for feedback to the controls system (described below) on time scales that are short in comparison to the tens of milliseconds needed for a single 2-D scan over the target area.

In various embodiments, the DMC 120 may comprise: a 2-D array of small gaseous ionization chambers; a gaseous tracking detector, such as a gas electron multiplier with fast electronic readout, combined with ionization chambers; a gaseous or thin plastic scintillator detector with fast position-sensitive readout; or any other analogous detector type or combination of detector types that provides the aforementioned capabilities.

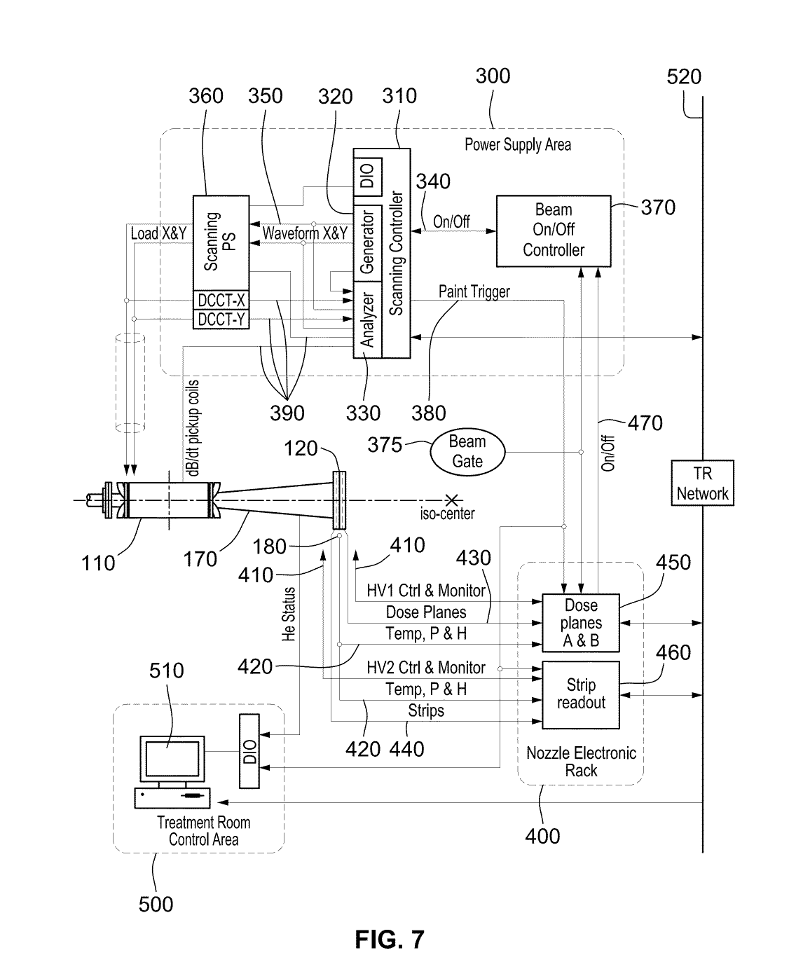

Referring now to the invention in more detail, in FIG. 7 there is shown a schematic diagram of a radiotherapy system that includes the fast scanning nozzle. The radiotherapy system is separated into a scanning controls module 300, a dose monitoring controls module 400 and a treatment room control area 500 containing the nozzle control computer 510. These major components communicate with one another via some direct connection digital and logic signals, but also via information transported on the treatment room network 520.

The scanning controls module 300 comprises a dedicated field programmable gate array (FPGA) controller 310 coupled to a signal generator 320 and a signal analyzer 330. The X-Y scan pattern along with the intensity modulation profile is loaded into the FPGA 310 as a 3-D array of numerical values. If the logic input 340 to FPGA 310 indicates beam on status, a dose painting cycle may be initiated, whereupon the generator module 320 will transmit the analog outputs 350 to the scanning magnet power supply 360 according to the numerical values in the 3-D array. The beam on/off controller 370 may incorporate a beam gate 375 that facilitates synchronization of irradiation with a patient's breathing cycle.

When a dose painting cycle is started, the FPGA controller 310 will generate a paint trigger signal 380 to transmit to the dose monitoring controls module 400 and to the nozzle control computer 510. Usage of a single 3-D array enforces synchronization of the scanning and intensity modulation processes. The FPGA controller 310 will sequentially execute each row of values in the 3-D array, then loop back and restart from the first row, repeating this repainting process until the prescribed dose is delivered at a given depth layer. A new 3-D array will be loaded for the next depth layer and the process will be repeated until the entire target volume is treated.

Still referring to FIG. 7, the second critical function of the scanning controls module 300 is monitoring the safety of the scanning process. This function is implemented in the signal analyzer 330 that monitors feedback signals 390 from the scanning magnet power supply and scanning magnet sensors. The accuracy of the scanning process is monitored by comparing the requested excitation of the scanning magnet 110 with feedback signals from the scanning magnet 110. The feedback signals include, but are not limited to, signals from Hall probes or equivalent devices that monitor the strength of the magnetic field inside the scanning magnet 110 and current sensors monitoring the output of the scanning magnet power supply 360. The FPGA controller 310 also provides output signals 340 that can interlock beam delivery into the nozzle 100 in case of failures in the scanning magnet 110 or its power supply 360 that are registered in the signal analyzer 330. The same signal analyzer 330 can accommodate other inputs, for example, from an optical system monitoring the patient's position, so that beam delivery can be interrupted if the patient moves by an amount above a chosen threshold distance.

The dose monitoring controls module 400 controls the Dose Monitor Chamber 120 via high voltage control and monitoring cables 410, monitors its temperature, pressure and humidity sensors 180 via signals 420, and processes its beam-induced output signals via cables 430 and 440. In one possible embodiment, the DMC 120 comprises ionization chambers including two integral plane electrodes and two electrodes with narrow X and Y strips. The integral plane electrodes collect the ions produced in the chamber gas by every proton delivered to the target; therefore, these two electrodes provide redundant information to the dose plane control module 450 about the absolute dose delivered to the treatment volume. The strip electrodes allow monitoring of the 2-D spatial profile of the dose delivery, and the transmission of this information to the strip readout module 460.

By synchronizing the strip readout electronics with the scanning process executed by the scanning controls module 300, the dose monitoring controls module 400 can determine the position, length and width of each one-dimensional line in each 2-D scan of the target. This information, transmitted on the treatment room network 520 to the nozzle control computer 510, will be used for a feedback system capable of correcting, after a few repaints, possible small beam misalignments in the fast scanning nozzle. Furthermore, strip electrodes also provide information about the intensity distribution along each scan line. This information will be used for monitoring the dose distribution accuracy. The dose monitoring controller 400 can also interrupt beam delivery to the nozzle, via logic signal 470, if dose delivery safety checks fail, permitting, for example, changes to the implementation plan for subsequent target repaints or resumption of an interrupted scan from the same 2-D position at which it was interrupted.

Due to the fast scanning nature of the proposed invention, the target area can be repainted as many as 100-200 times during a one-minute dose delivery process, e.g., 10 times per breathing cycle for 20 breathing cycles. A beam fluctuation or an error on a single paint will then result in dose perturbations smaller than 1%, which is well within common dose accuracy standards in radiation therapy. This feature of the fast scanning nozzle 100 and control systems 300, 400 and 500 improves the robustness and safety of the dose delivery process against various hardware and/or software failures.

Furthermore, the fast rescanning of each depth layer, as described herein, brings a number of advantages in comparison with the discrete spot scanning systems presently available commercially. Some of these advantages include the following.

1) The fast scan process does not create hot and cold spots in the target dose distribution. The target tumor will be essentially stationary during any single paint. Multiple repaints may be combined at slightly different target positions to deliver the full dose, and this may wash out dose gradients to a small extent, but will not lead to areas of significant over- or under-dosage, such as are seen for discrete spot beam scans in FIGS. 4 and 5.

2) Effects of beam misalignment are minimized without adding treatment time. Dose monitors will be used to implement a position feedback system capable of correcting possible small beam misalignments after the first few paints. Since each of the multiple repaints will deliver a small fraction of the full dose, the remaining repaints will minimize the overall dose perturbation that might be caused by an early beam misalignment.

3) Dynamic range demands on the beam intensity control and on the in-beam dose monitoring systems will be relaxed. By subdividing the dose into small repaint fractions, the ratio of maximum to minimum instantaneous dose delivery rates during a patient treatment will be considerably reduced. The higher doses needed for distal than for proximal depth layers will be achieved by using more repaints for the distal layers.

4) The fast rescanning can be easily combined with beam gating. An integer number of paints will be delivered in each “gate on” period synchronized with breathing mode, as in a CT scan, when the target is at a particular phase or position. If more repaints are needed to complete dose delivery to a given depth layer, this process will be repeated in subsequent gating periods, when the target has returned to nearly the same position.

The fast scanning nozzle will thus ameliorate several present limitations of pencil beam scanning approaches, thereby improving the precision of Intensity Modulated Ion Beam Therapy treatments, without increasing treatment time in comparison with currently available systems. Embodiments of the present invention emphasize, and provide for, critical features needed to take best advantage of continuous scanning. Most important among these new critical features are: the high speed of scanning needed to implement the “many-repaints scheme” that minimizes limitations associated with normal organ motion, with potential hardware and software problems in the delivery system, and with the high dynamic range demands on beam controls and dose monitors; the combination of two-dimensional (2-D) scanning in a single fast, combined-function scanning magnet that improves the accuracy of treatment implementation by providing a common source point for beam deflections in two orthogonal directions; the synchronization of fast beam scanning and fast dose monitor readout controls that improves the robustness of ion beam therapy treatments by facilitating mid-course feedback and corrections.

In summary, the advantages of the present invention include, without limitation: (1) a method and a system to facilitate two dimensional raster beam scans of depth layers within a tumor up to 25 cm×25 cm lateral dimension in scan times less than or comparable to 100 milliseconds; (2) the ability to scan continuously in two dimensions sharing a common source point for the beam deflection, improving the accuracy with which a treatment plan can be implemented; (3) the ability to subdivide dosage for a given depth layer in a pencil beam scan among multiple repaints, many of which can be carried out within a given patient breathing cycle; (4) a method and a system to avoid the hot and cold dose spots that can compromise a spot beam scanning approach for dose delivery to a target moving over patient breathing periods; (5) a feedback method for minimizing the impact of possible beam misalignments on ion beam dose delivery, without extending patient treatment times; (6) significant reduction of the dynamic range required of dose rate control systems and of dose monitoring detectors and electronics; (7) incorporation of dose monitors with millimeter-scale position resolution and response times to support millisecond-scale feedback to the nozzle controls; and (8) a controls system that synchronizes beam scanning and dose monitor readout controls to allow for optimized real-time safety assurance during dose delivery.

In a broad embodiment, the present invention is a fast scanning nozzle system to deliver intensity-modulated ion beam therapy radiation doses closely conforming to tumors of arbitrary shape, via a series of two-dimensional continuous raster scans of a pencil beam, wherein each scan takes no more than about 100 milliseconds to complete. In certain embodiments, the system includes: a fast, combined-function X-Y steering magnet; a rastering control system capable of adjusting the length of each scan line, continuously varying the beam intensity along each scan line, and executing multiple rescans of a tumor depth layer within a single patient breathing cycle; and an in-beam absolute dose and dose profile monitoring system capable of millimeter-scale position resolution and millisecond-scale feedback to the control system to ensure the safety and efficacy of the treatment implementation.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for irradiating a target volume with a charged particle pencil beam, the method comprising:
continuously scanning the pencil beam of charged particles over a two-dimensional (2-D) raster scan pattern;
applying length-variation for each scan line to conform to the 2-D raster scan pattern at a given depth;
applying pencil-beam-intensity variation along each scan line; and
completing multiple pencil beam scans of the 2-D raster scan pattern for each target depth layer of the target volume.

2. The method of claim 1, further comprising:
pausing the scanning upon completion of the scanning of each target depth layer in the target volume; and
changing the pencil beam energy value prior to scanning a next target depth layer.

3. The method of claim 1, further comprising:
measuring a position, length, and intensity distribution of each scan line; and
using the measurements to make feedback corrections to pencil beam position and pencil beam intensity for scanning subsequent scan lines or for subsequent repaints of the entire 2-D raster scan pattern for a given target depth layer.

4. The method of claim 1, wherein continuously scanning the pencil beam of charged particles comprises scanning the pencil beam along a scan line at a speed of at least 25 meters per second.

5. The method of claim 1, wherein continuously scanning the pencil beam of charged particles over a two-dimensional (2-D) raster scan pattern comprises scanning the entire 2-D raster scan pattern in 100 milliseconds or less.

6. The method of claim 5, further comprising gating the scanning of the pencil beam, wherein the gating is timed with respect to a patient's breathing cycle, so that an integral number of repaints of the 2-D raster scan pattern for a given target depth layer can be completed within each gating period.

7. The method of claim 1, wherein continuously scanning the pencil beam of charged particles over a two-dimensional (2-D) raster scan pattern comprises continuously scanning the pencil beam of charged particles over a two-dimensional (2-D) raster scan pattern using a fast scanning nozzle with scanning magnet.

8. The method of claim 1, further comprising measuring a dose distribution as a function of position along each scan line such that a measurement of absolute dose is accurate to within 2%, and a measurement of pencil beam spatial position is accurate to within two millimeters in each of two lateral dimensions.

9. The method of claim 8, further comprising synchronizing scanning of the pencil beam with the measuring of dose distribution.

10. The method of claim 8, further comprising interrupting pencil beam operation if the absolute dose measurement indicates that an actual dose delivery is outside of a predetermined range of acceptable values.

11. The method of claim 1, further comprising:
monitoring electric current drawn by the scanning magnet;
monitoring magnetic field strength of the scanning magnet;
monitoring patient position with respect to pencil beam position; and
discontinuing pencil beam operation if any one of the electric current, magnetic field strength, and patient position deviates from a predetermined range of acceptable values.

12. A system for delivering targeted ion beam therapy to a target volume, the system comprising:
a fast-scanning nozzle for targeting an ion beam, the fast-scanning nozzle having a scanning magnet configured to deflect the ion beam in two dimensions; and
a scanning magnet controller configured to control the fast-scanning nozzle to provide continuous scanning of the ion beam over a 2-D raster scan pattern at a first target depth layer of the target volume such that multiple scans of the 2-D raster scan pattern are performed, and further configured to control the fast-scanning nozzle to make multiple ion-beam scans of 2-D raster scan patterns for each of a plurality of target depth layers of the target volume other than the first target depth layer.

13. The system of claim 12, wherein the fast-scanning nozzle and scanning magnet are configured to deflect the ion beam in two perpendicular lateral dimensions at speeds exceeding 25 meters per second, such that the two perpendicular lateral beam deflections have identical source-to-axis distances.

14. The system of claim 12, wherein the fast-scanning nozzle further comprises a nozzle housing surrounding the scanning magnet, the housing having an ion beam entry window at a first end of the housing, and an ion beam exit aperture at a second end of the housing opposite the first end.

15. The system of claim 14, wherein the ion beam exit aperture is disposed in a retractable housing projection.

16. The system of claim 15, wherein the retractable housing projection includes a holder for patient-specific apertures or compensators.

17. The system of claim 14, wherein the fast-scanning nozzle further comprises a beam monitoring ionization chamber adjacent to the ion beam entry window, the beam monitoring ionization chamber configured to measure the size, position, and intensity of the ion beam after it passes through the ion beam entry window, and to provide the measurement data to the scanning magnet controller.

18. The system of claim 17, wherein the scanning magnet controller is configured to make feedback corrections to ion beam position and intensity based on the measurement data from the beam monitoring ionization chamber.

19. The system of claim 14, wherein the fast-scanning nozzle further comprises a dose monitoring chamber downstream of the scanning magnet and upstream of the ion beam exit aperture, the dose monitoring chamber configured to provide data, regarding dose delivery and ion beam spatial position, to the scanning magnet controller.

20. The system of claim 19, wherein the dose monitoring chamber comprises:
a position-sensitive array of gaseous ionization chambers;
or a gaseous tracking detector coupled to position-insensitive ionization chambers;
or a scintillation detector with position-sensitive readout.

21. The system of claim 19, further comprising one or more sensors disposed in the nozzle housing proximate the dose monitoring chamber, the one or more sensors configured to sense one of temperature, humidity, and pressure.

22. The system of claim 19, wherein the fast-scanning nozzle further comprises a light projection mirror disposed in the nozzle housing downstream from the dose monitoring chamber, the light projection mirror configured to align the target volume with the fast scanning nozzle.

23. The system of claim 12, further comprising an energy modulation unit configured to vary the energy of the ion beam before it enters the fast scanning nozzle.

24. The system of claim 12, wherein the scanning magnet controller controls a safety interlock configured to:
shut off the ion beam if a dose measurement indicates that an actual dose delivery is outside of a predetermined range of acceptable values; and
shut off the ion beam if any of one or more sensors, monitoring one of electric current drawn by the scanning magnet, magnetic field strength of the scanning magnet, and patient position with respect to pencil beam position, senses that one of the electric current, magnetic field strength, and patient position is outside of a predetermined range of acceptable values.
